(19) United States
(12) Patent Application Publication
(10) Pub. No.: US 2005/0137470 A1
(43) Pub. Date: Jun. 23, 2005

(54) Method and apparatus for low blood glucose level detection

(76) Inventor: Robert D. Rosenthal, Silver Spring, MD (US)

Correspondence Address: ROTHWELL, FIGG, ERNST & MANBECK, P.C., 1425 K STREET, N.W., SUITE 800, WASHINGTON, DC, 20005, US

(21) Appl. No.: 11/006,546

(22) Filed: Dec. 8, 2004

Related U.S. Application Data
(60) Provisional application No. 60/527,292, filed on Dec. 8, 2003.

(51) Int. Cl. A61B 5/00

(52) U.S. Cl. 600/316

(57) ABSTRACT

A method and system for detection of potentially undesirable and/or dangerously low levels of blood glucose based on heart rate measurements in conjunction with initial calibration of blood glucose levels, and continuous monitoring of heart rate and estimation of blood glucose levels during periods of sleep.

### CROSS-REFERENCE TO PROVISIONAL APPLICATION AND CLAIM FOR PRIORITY UNDER 35 U.S.C. § 119(e)

This application claims the benefit of the filing date of Provisional Application Ser. No. 60/527,292, filed on Dec. 8, 2003.

### BACKGROUND OF THE INVENTION

1. Field of the Invention

This invention relates generally to medical diagnostics and in particular to methods for measuring certain blood analytes, such as blood glucose.

2. Background and Conventional Art

People who have diabetes are constantly attempting to keep their blood glucose level within a small acceptable range. If the blood glucose level becomes too high (a condition called hyperglycemia), damage to various capillaries in the body over a period of time could cause serious diabetes complications. Some of these potential complications are blindness, loss of limbs, kidney failure, and heart failure. If the blood glucose level falls too low (a condition known as hypoglycemia), the brain becomes starved for energy. This could cause loss of consciousness, and even death.

To control their blood glucose level, people with diabetes normally use “finger stick” technology to determine their current blood glucose level. To make such measurements, a body part (e.g., a finger or the forearm) is punctured and a small amount of bodily fluid (either blood or interstitial fluid) is placed on a chemically laden disposable strip and then measured for glucose content by a portable meter. Persons with diabetes are normally advised to test themselves a minimum of four times per day, as it is recognized that blood glucose levels vary throughout the day and during overnight sleep. The variations are particularly dangerous for Type 1 diabetics, who may, during nighttime sleep, fall into a life threatening hypoglycemic state.

In the last few years, several technologies have become available that provide methods for tracking blood glucose level at any time during the day, and in particular, during the sleep cycle. One such product, sold under the commercial name GlucoWatch (Cyngus, Inc.), uses “reverse iontophoresis technology” to provide the measurement. To accomplish this, a specialized absorbent pad called an “Auto Sensor” is placed under a specially developed watch worn on the arm, which uses an electrical current to cause interstitial fluid to be drawn into the pad. The watch system then automatically analyzes the glucose content in the pad and provides an estimate of blood glucose level approximately once every ten minutes (see U.S. Pat. No. 6,561,978).

To obtain proper measurements, the GlucoWatch system requires a new Auto Sensor pad to be used every thirteen hours at a cost of approximately $5 each. Moreover, it has a number of other requirements and/or limitations that may interfere with the measurement, such as the possible need for shaving the arm to allow proper seating of the Auto Sensor, the potential for irritation of the skin causing a rash or blisters, and the inability for the measurement to be made if the arm is perspiring.

A second method for determining continuous blood glucose level involves inserting a small sensor beneath the skin of the abdomen. This solid state glucose sensor is attached to an external Continuous Glucose Monitor. However, because of the body's reaction to the sensor, it is limited to use only for a few days. Moreover, the replaceable sensors are expensive, at more than $50 each.

Additionally, there are many patents in the prior art that illustrate that non-invasive measurements could be performed using near-infrared techniques (see e.g., U.S. Pat. Nos. 5,028,797 and 5,077,476). A difficulty with these approaches is that they require the use of relatively expensive instrumentation, and are very sensitive to disturbances. Moreover, they are not designed for continuous measurement.

What is needed is a low-cost method for continuously determining blood glucose level in the low glucose range that: (1) does not require any expensive expendable items and, (2) provides an accurate measurement of blood glucose levels. This need is solved by the present invention.

### SUMMARY OF THE INVENTION

The present invention solves the need identified above by providing a method and system for detection of potentially undesirable and/or dangerously low levels of blood glucose based on heart rate measurements in conjunction with initial calibration of blood glucose levels, and continuous monitoring of heart rate and estimation of blood glucose levels during periods of sleep.

In particular, according to one aspect of the invention, a method is provided for detecting a potentially undesirable low level of glucose in the blood, which includes the steps of taking an accurate measurement of blood glucose level, taking an initial measurement of heart rate within a predetermined amount of time from the taking of the accurate measurement, periodically monitoring heart rate over a predetermined extended period of time, and estimating blood glucose level as a function of the periodically monitored heart rate, initial measurement of heart rate, and accurate measurement of blood glucose level.

According to another aspect of the invention, a system is provided for detecting a potentially undesirable low level of glucose in the blood, including an instrument for taking an accurate measurement of blood glucose level, an instrument for taking an initial measurement of heart rate within a predetermined amount of time from the taking of the accurate measurement and periodically monitoring heart rate over a predetermined extended period of time, and a device for estimating blood glucose level as a function of the periodically monitored heart rate, initial measurement of heart rate, and accurate measurement of blood glucose level.

### BRIEF DESCRIPTION OF THE DRAWINGS

FIGS. 1 and 2 are charts showing relationships between heart rate and blood glucose level;

FIGS. 3A-3C are graphs showing relationships between predicted blood glucose levels and measured heart rate over time, and correlation between predicted levels and measured reference levels;

FIGS. 4-6 are graphs illustrating ECG data and correspondence between ECG data and a second derivative of measured pulse rate.

FIG. 7 is a graph showing the power spectral density of heart beat frequencies;

FIGS. 8 and 9 are graphs showing slow-wave heart rate activity during a period of sleep;

FIG. 10 is a graph of the output over time of a pulse monitoring instrument according to one embodiment of the invention;

FIG. 11 is a diagram of a system for detecting blood glucose level according to one preferred embodiment of the invention;

FIG. 12 is a flow diagram showing a procedure for obtaining and analyzing pulse data in conjunction with blood glucose level estimation according to one preferred embodiment of the invention;

FIGS. 13A-13B, 14A-14B, 15A-15B, 16A-16B, and 17 are waveform charts illustrating heart rate data acquisition, processing and analysis according to principles of the present invention.

### DETAILED DESCRIPTION OF THE PREFERRED EMBODIMENTS

In U.S. Pat. No. 6,477,392 to Honigs, incorporated herein by reference in its entirety, it is disclosed that there is a weak but meaningful correlation between heart rate and blood glucose. This relationship, in general, has a correlation of less than 0.5. In co-pending patent application Ser. No. 10/387,845 to Rosenthal, also incorporated herein by reference, the use of heart rate in combination with other parameters for measuring blood glucose is expanded upon. However, the contribution of heart rate as one of the multiple regression variables remains quite small.

Method

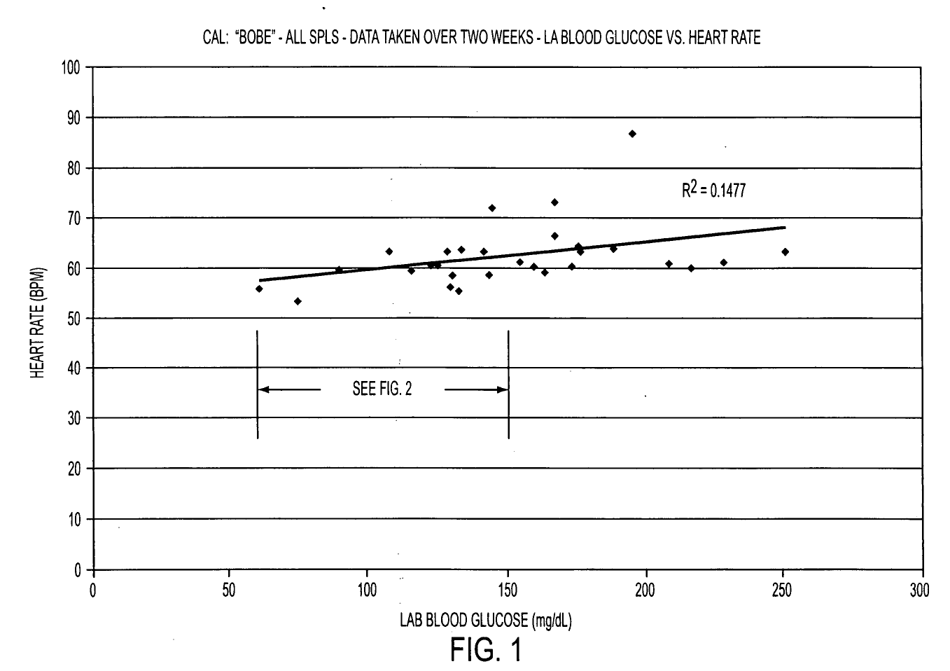

As shown in FIG. 1, there is a weak relationship between blood glucose level and heart rate over a wide blood glucose range. This data is from a single individual during a relatively long-time test (on the order of weeks). As indicated in FIG. 1, the Coefficient of Determination (i.e., R-squared) over this range is only 0.1477.

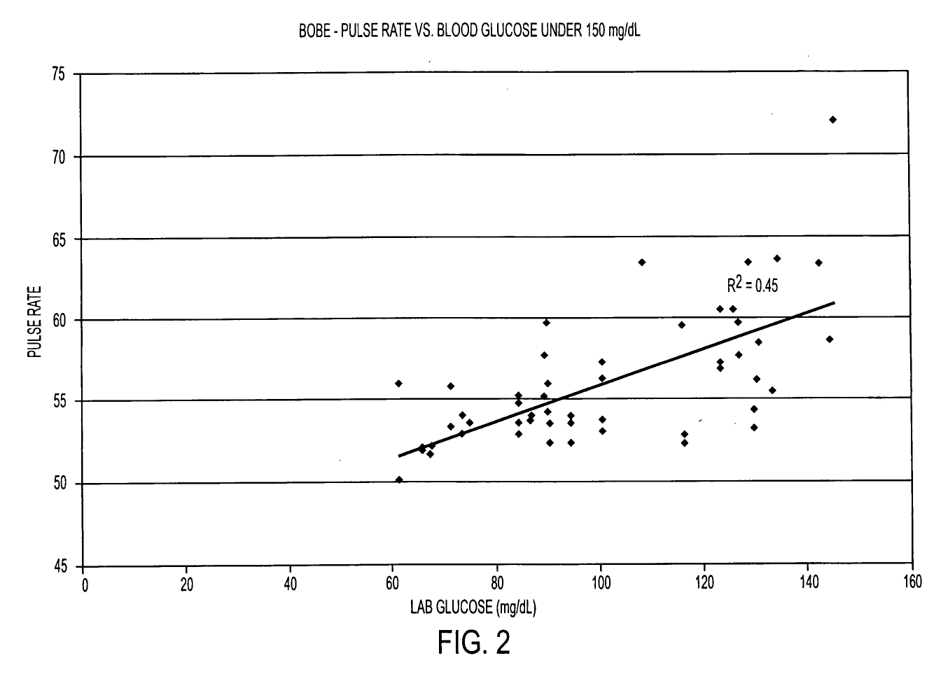

However, if only the lower glucose levels (e.g., below 150 mg/dL) are considered, a much more distinctive and meaningful relationship between blood glucose level and heart rate is demonstrated as shown in FIG. 2. But even in this low glucose range, the R-squared of heart rate to blood glucose is still only 0.45, which is not as high as would be desirable for accurate quantitative measurement of the blood glucose level.

In researching this phenomenon, two parameters that influence R-squared were discovered. First, if the body is relaxed and at rest—for example when sitting relatively still or sleeping—there is a much higher correlation between heart rate and blood glucose during such basal heart rate (“BHR”) conditions. The second discovery is that there can be day-to-day differences in the BHR.

This phenomenon was evaluated by having insulin-dependent diabetics attached to a two wavelength optical finger clip unit of the type typically used in pulse oximeters, for a period of approximately three hours. For this test a special set of electronics was attached to the pulse oximeter's finger clip unit to provide a more accurate measurement of heart rate. Heart rate is usually measured in terms of beats per minute, for example, 60 beats per minute. However, for this test the heart rate was recorded at a ten times higher sensitivity, or in terms of tenths of beats per minute, e.g., 60.3 beats per minute.

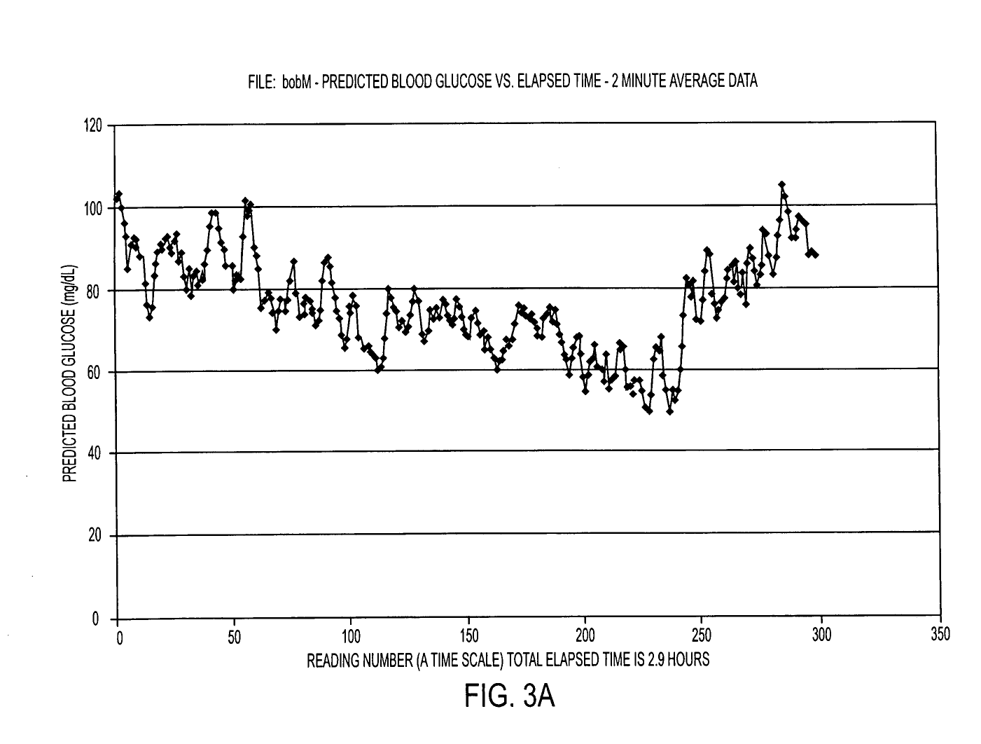

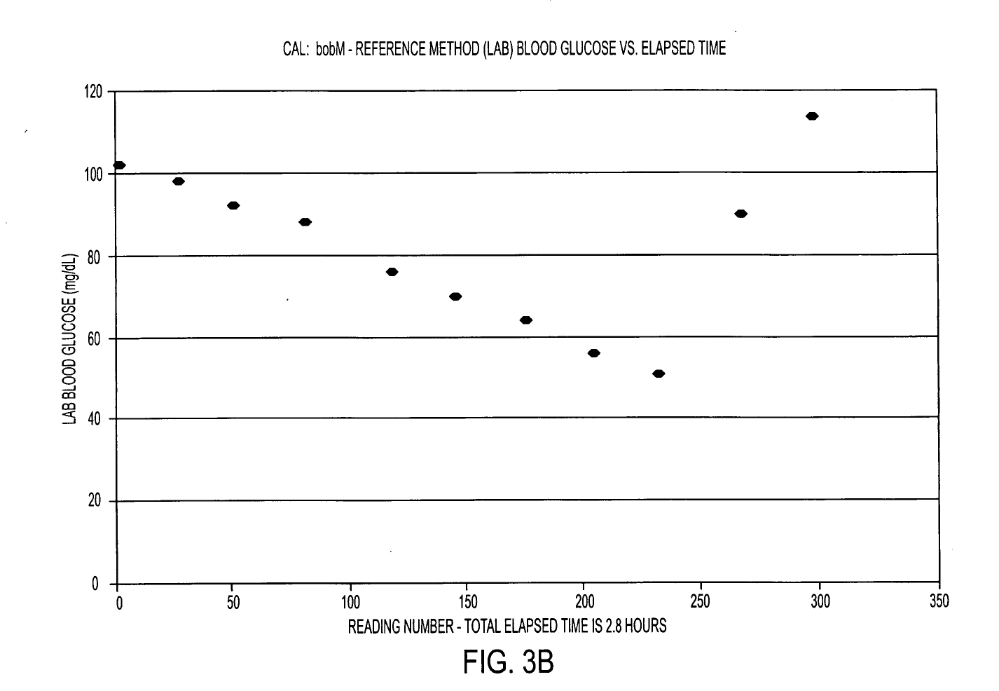

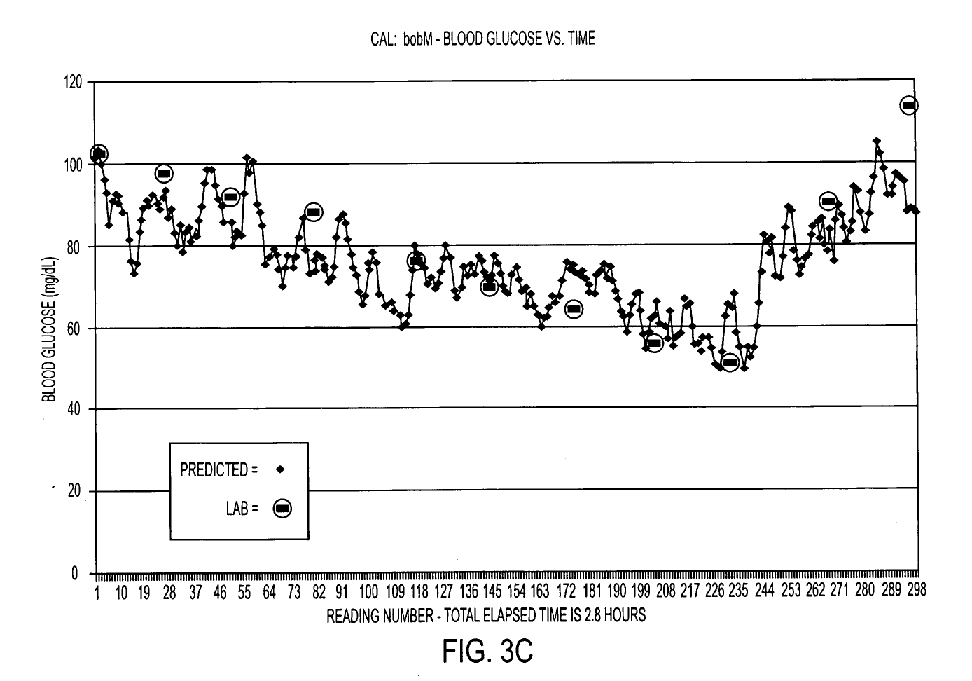

FIG. 3A illustrates a continuous predicted glucose measurement over a 2.8 hour time period for one diabetic individual based on heart rate. Every fifteen minutes during this test, finger stick measurements were performed and the blood glucose level was determined by taking the average of two One Touch Profile instrument measurements. This was considered the reference method (identified as “LAB” in FIG. 3B). In FIG. 3C, the lab values (FIG. 3B) are superimposed on the predicted values (FIG. 3A).

In deriving FIG. 3A, the lab value at the start of the test period was used to bias-adjust the heart rate/glucose instrument so that it read exactly the same as the One Touch Profile. Moreover, the calibration constant that multiplied the heart rate was derived from the relationship shown in FIG. 2; i.e., from data taken on the same individual approximately six months prior to the continuous glucose test. As shown on FIG. 3C there is good agreement between continuous glucose measurement based on heart rate and the lab values.

In summary, the relationship of the continuous glucose measurement to the lab value was determined using linear regression techniques. The equation was
Predicted blood glucose at any time=(initial blood glucose)+K(1)×(delta heart rate) (1)

Where:
“Initial blood glucose” is the finger stick reading (i.e., the “lab value”) at the start of the continuous blood glucose reading
K(1) is the slope from FIG. 2; and

“Delta Heart Rate” is the heart rate at any time minus the initial heart rate at the start of the test.

The data shown in FIG. 3A is for the average heart rate over each three minute period. No data was omitted or skipped in deriving this figure. It is recognized that this data could be improved with proper data filtering to eliminate heart rate measurements that are not at BHR. Such higher heart rates occur from body motion or by involuntary violent events (e.g., sneezing or coughing); or from physical exertion.

There are several limitations of using Equation 1 to determine blood glucose level. One of the limitations is what occurs while someone sleeps. Before describing the problem and its solution, some basic definitions are necessary.

ECG Analysis Versus Second Derivative of Optically Determined Heart Rate

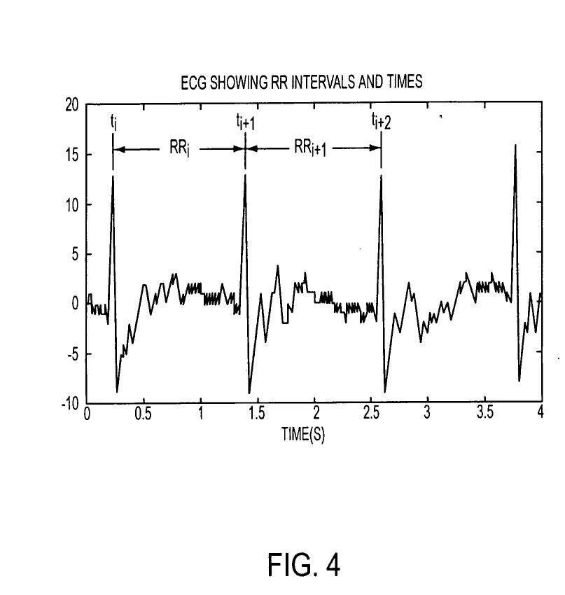

Electro Cardiogram (“ECG”) analysis is usually performed to determine the characteristics of the heart beat. FIG. 4 illustrates the ECG of a normal person during four heart beat cycles (the normal heart rate is approximately 60 beats per minute).

In the figure, the time of each heart beat is shown as t_{I}, t_{I+1}, and t_{I+2}. The heart rate (bpm) can be simply determined by counting the number of these “t” cycles that occur in one minute. FIG. 2 also defines the interval “RR” as the time between two adjacent heart beats. The literature shows that the variability of RR provides a powerful diagnostic tool for determining a large number of health characteristics.

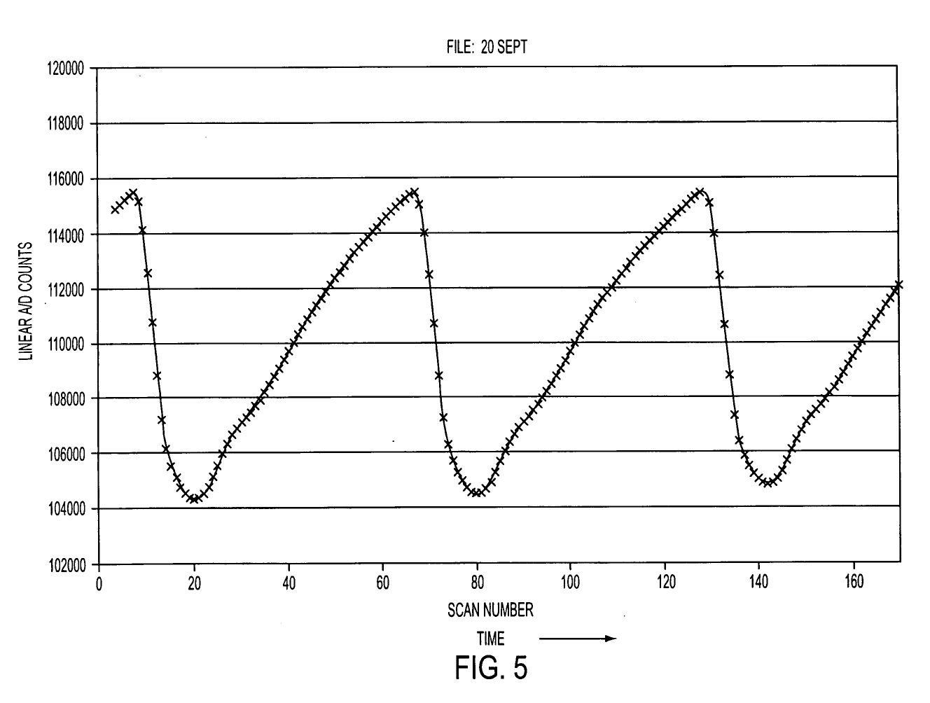

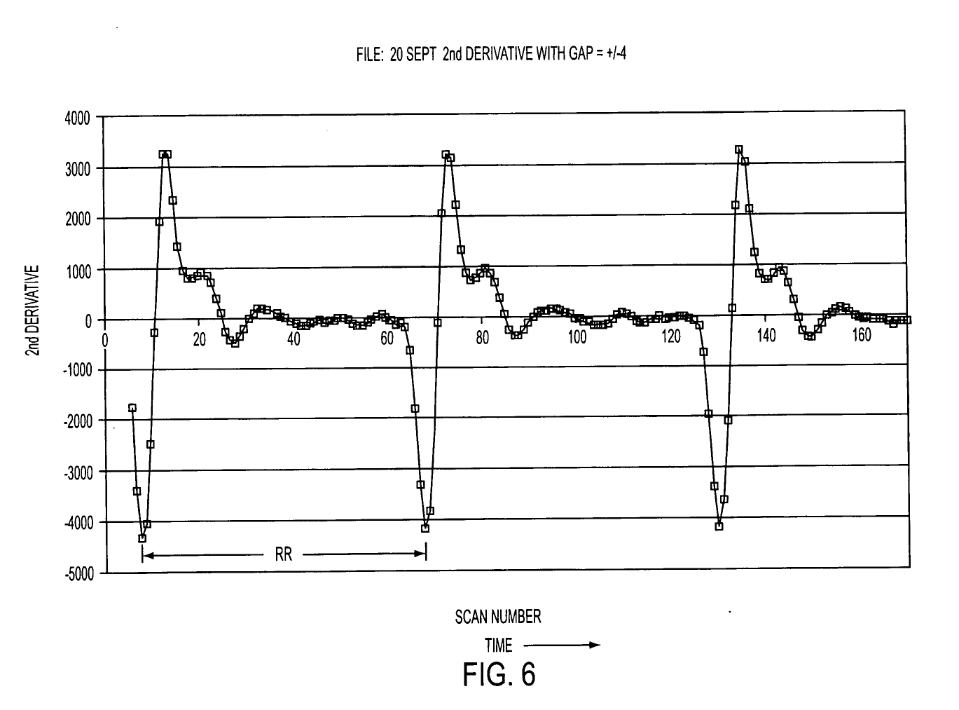

FIG. 5 shows typical heartbeats optically measured with a pulse oximeter optical fingertip sensor. FIG. 6 shows the second derivative of the waveform of FIG. 5. As shown, a distinct pattern of the pulse beat is produced by the second derivative, which is similar to the inverse of the ECG pattern shown in FIG. 4.

The second derivative approach eliminates variation due to baseline fluctuation in the basic heart rate measurement. Thus, the second derivative can provide not only a direct mode of determining heartbeat rate, but also can be used to determine the RR values.

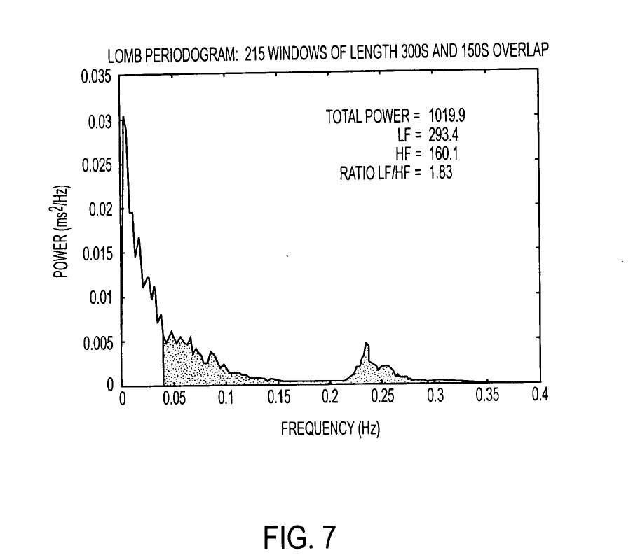

FIG. 7 illustrates another approach for analyzing the variability of RR (hereinafter called “Heart Rate Variability” or “HRV”). The figure is derived by converting the time-based coordinate system of FIG. 4 to the frequency domain (i.e., where the horizontal axis denotes the frequencies at which RR values occur).

As shown in FIG. 7, the HRV calculated over a two to five minute period is concentrated in two distinct different parts of the frequency domain—in the so-called Low Frequency (LF) range (0.04-0.15) and also in the High Frequency (HF) range (above 0.15). Heart Rate and the Heart Rate Variability are each important but separate indicators during sleep.

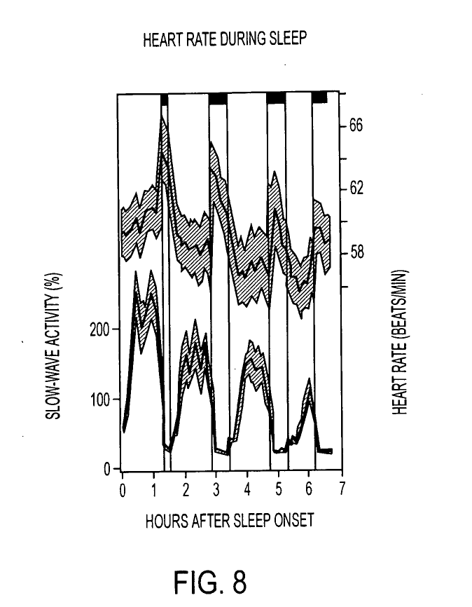

FIG. 8 summarizes heart rate data of 16 normal individuals taken during a full night's sleep. As shown, the sleep period is divided into distinct parts:
Non-rapid eye motion sleep (“NREM”), and
Rapid eye movement sleep (“REM”).
   The periods of REM are shown by the solid bars at the top of the figure.

The waveforms at the top of FIG. 8 represent the variation in Heart Rate for sixteen normal individuals as a function of how many hours they were asleep. It is noted that in the approximately six and one half hours of sleep there were four REM periods, the first one starting typically about an hour and a half after sleep was commenced.

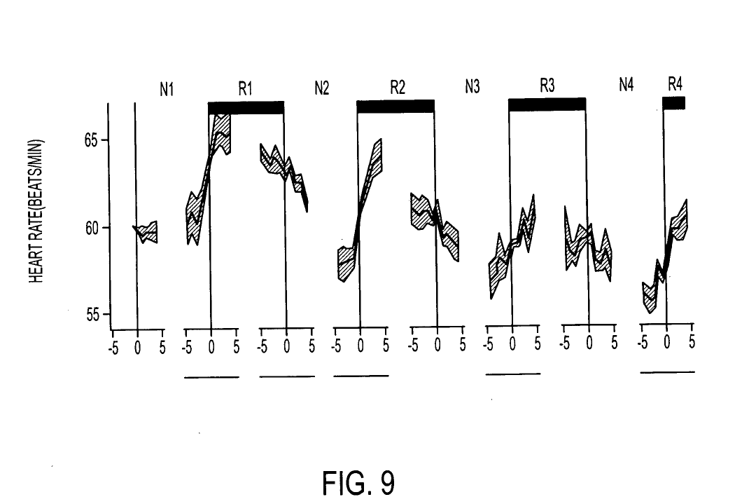

The bottom part of FIG. 8 represents the low frequency wave of the HRV as described previously. Here, it is noted that at the start of a REM stage there is a large increase in Heart Rate of approximately four or five beats per minute. Similarly, at the end of REM sleep there is a rapid decline in Heart Rate. These characteristics are more clearly shown in FIG. 9.

At the top of FIG. 9 the variable “N” indicates Non-REM sleep periods and the variable “R” indicates periods of REM sleep. The horizontal data covers five minutes prior to and five minutes after the start of each of these specific periods. Referring to the boundary between N1 and R1, a rapid increase occurs in Heart Rate from about 60 beats per minute to 65 beats per minute in roughly three minutes, which corresponds to a change of approximately 1.7 heart beats per minute. Previous research has provided the following information:
People who do not have diabetes experience a change in blood glucose level at a maximum rate of approximately 1 mg/dL per minute;
People with Type 2 diabetes experience a change in blood glucose level at a maximum rate of approximately 2 mg/dL per minute;
People with Type 1 diabetes experience a change in blood glucose level at a maximum rate of approximately 3 mg/dL per minute.

As derived from FIG. 2, the proportionality constant between heart rate and blood glucose is approximately 7.0. This would indicate a maximum change in blood glucose level of 1.7×7=12.0 mg/dL per minute, which is impossible because the maximum blood glucose level rate of change can be only about 3 mg/dL per minute.

Therefore, the abrupt Heart Rate changes as shown in FIG. 9 would provide a clear indication of entering a different sleep state. When these sleep state transitions occur, the non-invasive instrument that uses Equation 1 will cease to provide any measurement of blood glucose levels. Measurements will be restarted only after completion of the sleep state transition (e.g., in about four to seven minutes). Similar pauses in non-invasive measurements would occur on both the entry into and the exit from a sleep state.

According to another embodiment of the invention, instead of using the linear regression method as shown in Equation 1, a two-term multiple linear regression can be used. In this method, the first variable term remains the change in heart rate and the second variable term is the slow-wave activity (i.e., “LF”) of the heart rate variability as shown on the bottom of FIG. 8 or an equivalent term in the time domain. As shown FIG. 8, the slow-wave activity rapidly plunges to a low level as heart rate surges when REM sleep is entered. The Multiple Linear Regression approach thus allows measurement to continue even during the change in sleep state.

With either a Linear Regression non-invasive instrument or Multiple Linear Regression non-invasive instrument, it is important that the user obey the following restrictions for at least two hours prior to going to sleep. The user must not:
participate in unusual or intensive exercise
eat a major meal
smoke
drink beverages containing caffeine
drink alcoholic beverage

Additionally, if the simple instrument incorporating Equation 1 is used, the slow-wave activity of FIG. 8 (i.e., low frequency component of the frequency domain or its equivalent in the time domain) could be used to further define when not to make the blood glucose estimation as a function of Heart Rate.

Apparatus

The previously described methods of measuring blood glucose at low glucose levels are predicated upon achieving an accurate measurement of heart rate. There are many different technologies that are available to detect heart rate. For example, microphonic devices, optical devices (such as used on fingertip pulse oximetry sensors), electrical devices (e.g., ECG), or manual devices (e.g., a nurse's finger held on the inside wrist veins while observing a clock) all could be used to obtain a sufficiently accurate measurement of heart rate. Any of these techniques could be used provided that their sensitivity is enhanced to allow pulse rate per minute to be determined to one decimal place accuracy.

According to one preferred embodiment, pulse rate is determined using a simple low-cost optical approach. An LED or IRED and sensor are located on the wrist or a fingertip, directly touching the skin (similar to that described in U.S. Pat. No. 4,928,014). In one preferred embodiment, an IRED emitting light between 900 and 950 nanometers (e.g., Stanley AN501 IRED) and a low-cost silicon photo detector (e.g., Hamamatsu Part #S23876-45K) can be used for such “interactance” measurements. The IRED and detector are both in contact with the skin, to prevent any light from being reflected from the surface of the skin to the detector. The only light received by the detector is scattered light that has entered into the wrist or fingertip and scattered by the flash in a direction returning to the detector.

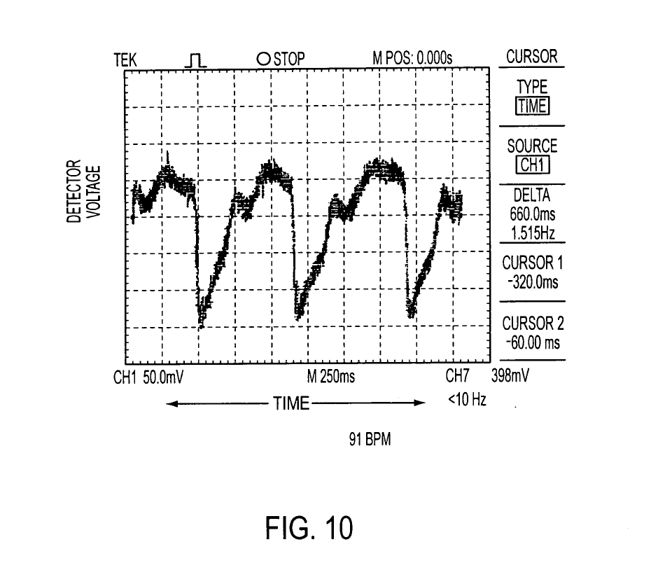

FIG. 10 is a typical pulse rate versus time plot using such type of wrist interactance optical system. As shown in FIG. 10, the pulse rate is clearly distinguishable and can be resolved to the required 0.1 beats per minute resolution. In the above approach, the IRED is constantly illuminated at a very low light level to allow operation from a battery power source. The optical energy that interacts with the body is totally non-ionizing and is intrinsically safe.

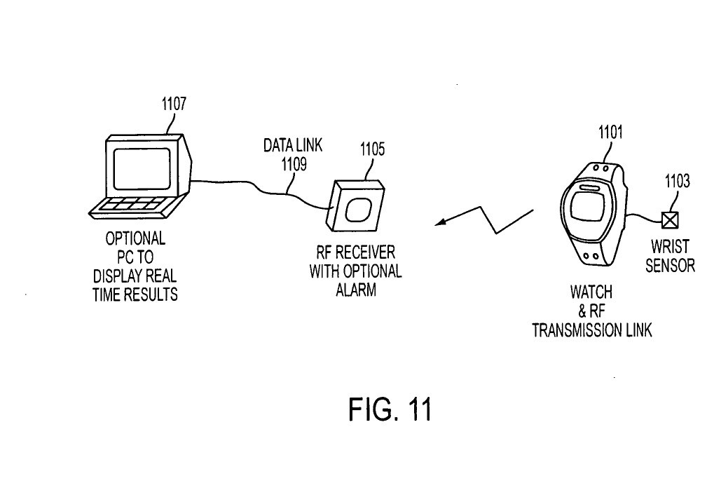

In one preferred embodiment as shown in FIG. 11, the wrist sensor 1103 is wired to a watch-type device 1101 (in fact, the watchband that holds the watch may contain the optical sensor). The watch-type device 1101 (hereinafter called “Watch”) contains a microprocessor with sufficient computation capacity and storage memory to interpret the heart rate data and to provide a direct readout of blood glucose using calibration constants as previously described.

The Watch 1101 contains an LCD display that shows the continuous blood glucose level (provided that the blood glucose level is below 150 mg/dL), and also may include a second display containing a real time clock (providing actual time). The Watch 1101 also includes an A/D converter, a LCD driver circuit, as well as sufficient RAM and non-volatile memory to store measurements covering at least a fourteen hour period.

The Watch 1101 may also contain a low-powered RF transmitter that is able to send measured blood glucose level data to a remote receiver 1105. The receiver 1105 can transfer the data via a data link 1109 to a PC 1107 or other type of computer where a software program converts the data to a continuous real-time graphical display of the blood glucose level. As part of the program, a low glucose alarm may sound, thereby awaking either the person being monitored or, if the individual is a child, awaking the child's parents. The low glucose alarm indicates the onset or existence of a potentially dangerous condition. Similarly, an adjustable alarm level can be built into the Watch 1101 allowing it to sound an alarm when the user's blood glucose level is low.

One of the most critical times for people with diabetes is when they are about to go into a hypoglycemic state during sleep. Thus, the alarm should have sufficient volume to wake a person even if the Watch 1101 may be muffled, for example by virtue of the arm wearing the Watch being under a pillow.

To compensate for body changes over time (e.g., during the night), at the start of any measurement cycle, the person puts on the Watch and presses a START button. After approximately two minutes, the Watch will prompt the person to do a conventional finger stick blood glucose measurement. The finger stick result is then entered into the Watch, as the bias correction term in Equation 1, and thereby allowing from that point on, the continuous glucose monitor will accurately predict low glucose levels.

A remote receiver also can be used as an alarm without a PC, so that a parent can be alerted to a potentially dangerous low-level blood glucose situation of a child.

The Watch can be powered by a rechargeable battery with sufficient capacity to run the system for approximately fourteen hours between recharges. This will allow the Watch to provide continuous data during nighttime sleep.

Data Analysis

The non-invasive blood glucose instrument of the present invention is simple in concept and implementation compared to typical optical measurement devices. For example, typical optical measurement devices require some type of “zero adjustment” to avoid drifts of the optical system. However, because the measurement being taken is of pulse rates and not of absolute optical measurement values, such zero adjustment (sometimes called “standardization”) is not required for the application of the present invention. Secondly, most optical measurement instruments require periodic calibration measurements at intervals with the light shut off to compensate for drifts of electronic components such as amplifiers, and other optical elements. Again, because no optical measurements are being taken in absolute terms such “dark measurements” are also not needed.

The third typical requirement of optical measurement instruments is that the data be converted into logarithmic form where the optical data is equal to the logarithm of one divided by the relative energy. Again, because no absolute measurements are being performed, there is no need for conversion of data to logarithmic form. The measured data instead is simply converted to digital data by a standard analog-to-digital converter, in terms of A/D “linear counts.” This is all that is required for quantitative measurement.

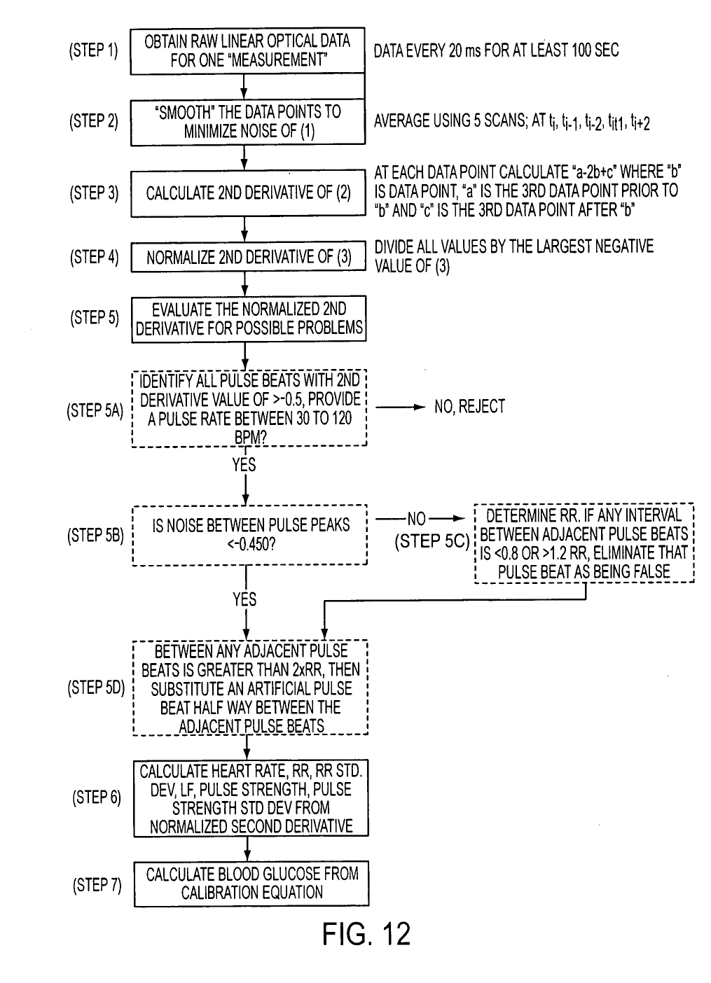

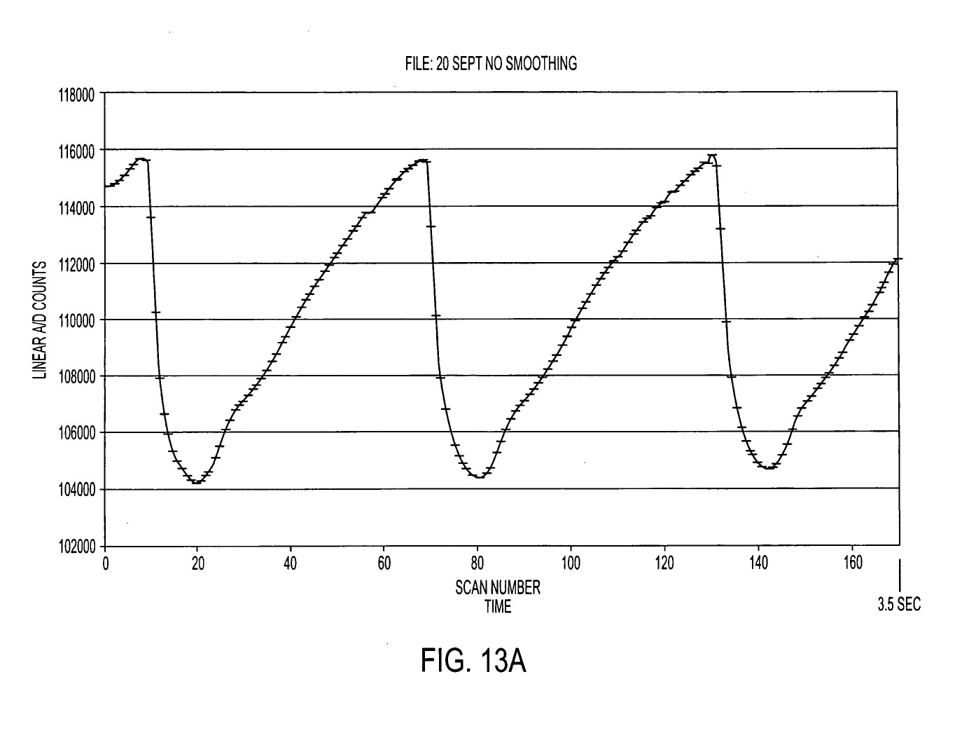

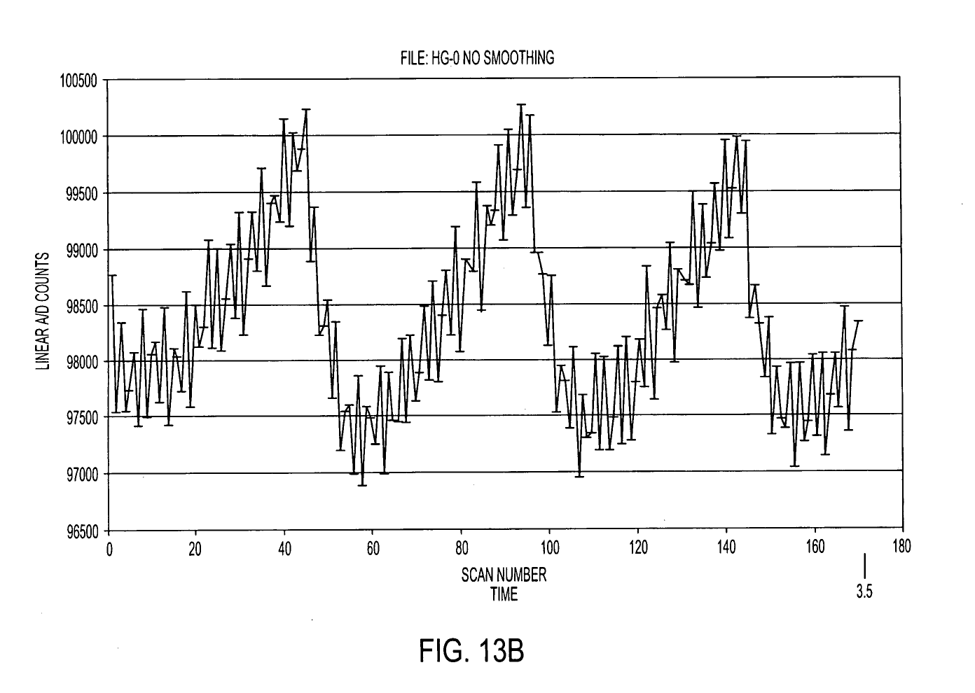

FIG. 12 is a flow chart showing a data analysis procedure according to one preferred embodiment of the present invention. The procedure is subdivided into seven major steps. In Step 1 raw optical data is obtained. FIG. 13A shows an example of raw optical data for an individual where the raw optical data is relatively noise free, and FIG. 13B shows more typical data where the raw data measurement has considerable noise. For convenience, both of these figures are shown limited to their first 3½ seconds so that the noise would be easily visible.

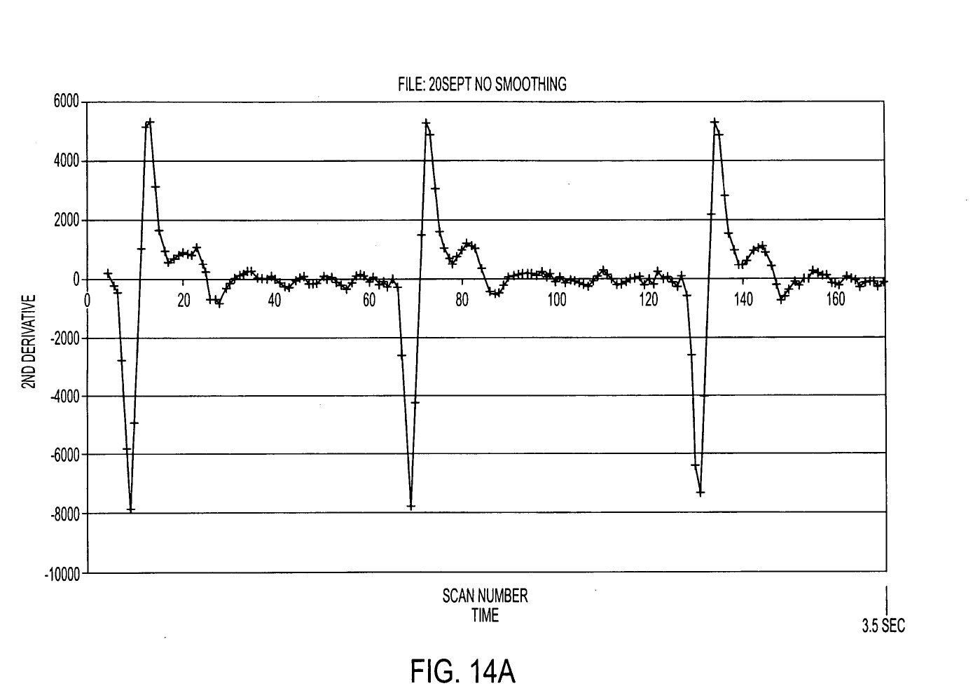

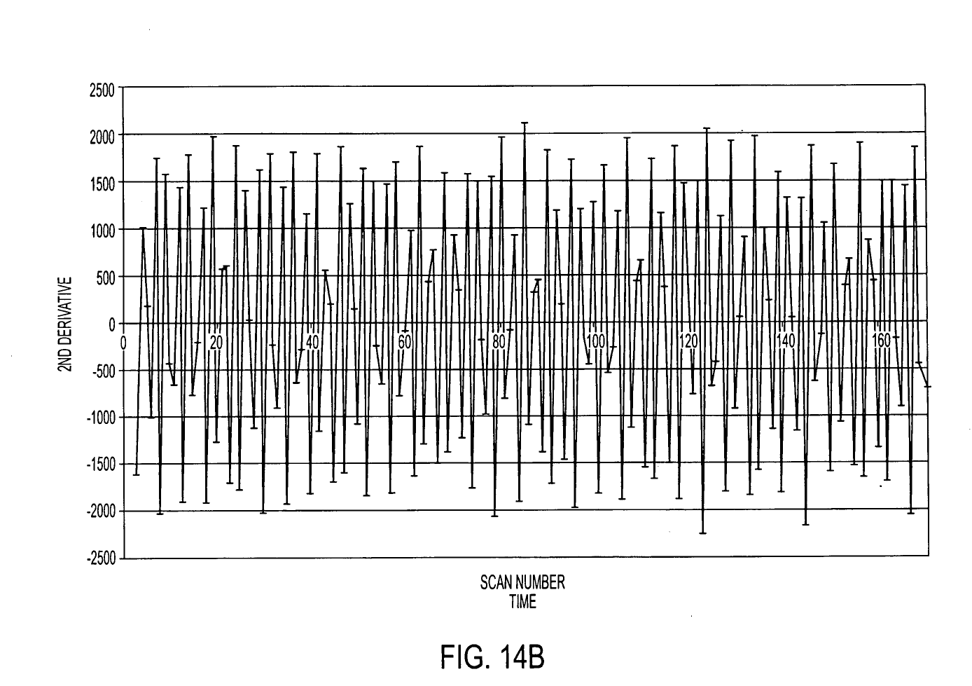

FIGS. 14A and 14B present the 2^{nd} derivative of FIGS. 13A and 13B respectively. As seen, the 2^{nd} derivative data of FIG. 14B is essentially worthless due to the noise in the linear A/D count data. Therefore, at Step 2 of FIG. 12, the raw optical data is smoothed, such as by taking a moving average over a number of data points such as 5, which effectively eliminates the noise.

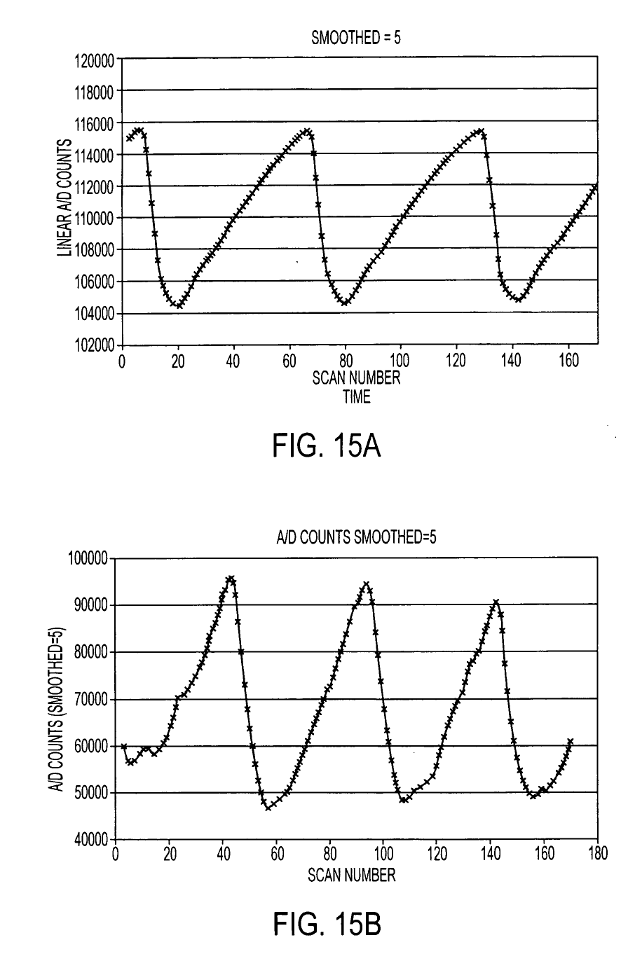

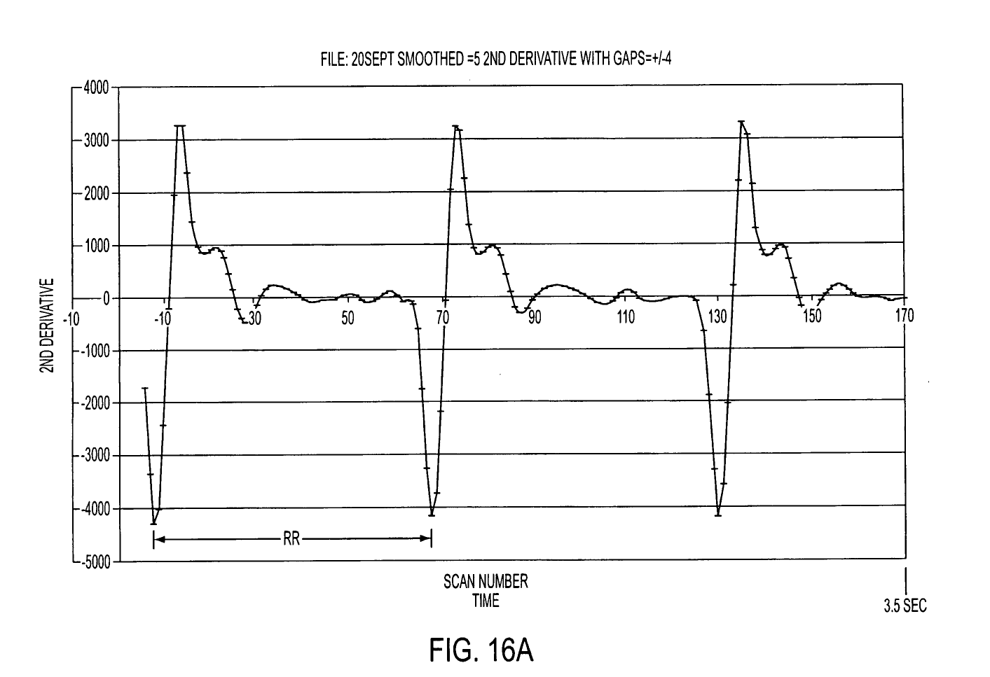

FIGS. 15A and 15B presents the same data as FIGS. 13A and 13B except that the data has been “smoothed” by averaging of A/D counts over five adjacent scans at each data point. A preferred method is to average the A/D counts at the current data point with the data of the two preceding scans and the two following scans. An alternate method is to average the current data point with the four preceding scans. As shown in FIGS. 15A and 15B, the data for both individuals that are represented in FIGS. 13A and 13B are quite noise free and usable for further analysis.

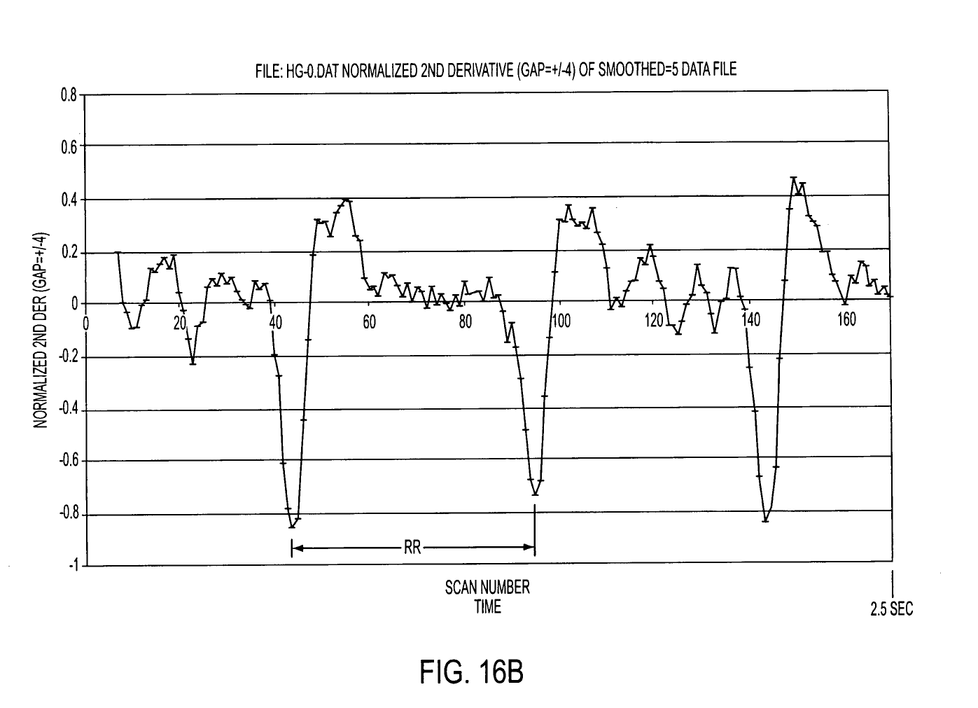

FIGS. 16A and 16B respectively show the second derivative of the smoothed data of FIGS. 15A and 15B. This step is performed at Step 3 of FIG. 12. As shown, the second derivative provides a good resolution of the time of each pulse signal. Moreover, taking the second derivative eliminates shifts in the baseline which are common in pulse measurement.

The second derivative can be calculated using the equation bracket [a−2*b+c] where “b” is the A/D count at the Scan Number (i.e., the time) of interest, “a” is the A/D count of the third Scan Number prior to “b,” and “c” is the A/D count at the third Scan Number (i.e., time) after “b” (commonly identified as the second derivative with a gap =+/−4).

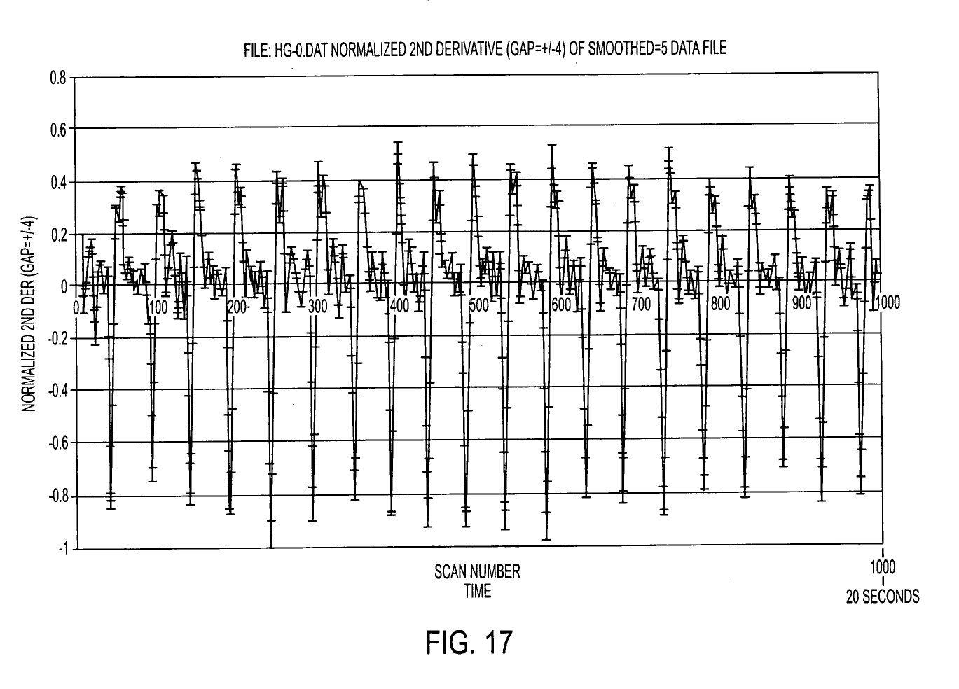

At Step 4, the second derivative data is normalized to eliminate the variability between optical scans of different individuals. This is accomplished by dividing all the second derivative values during each measurement by the largest A/D count of any pulse signal during that measurement. This will force the maximum pulse signal during any measurement to be −1.0. FIG. 17 shows the normalized 2^{nd} derivative data from FIG. 16B, for 20 seconds of measurement. Among other advantages, the normalized 2^{nd} derivative provides a means of calculating the time between pulse beats (“RR”). At step 5A, a validity test is performed to insure that the calculated heart rate is realistic; e.g., is between 30 and 120 beats per minute.

Experimentation has demonstrated that the normalized second derivative value of the pulse beats between different individuals varied between −0.619 and −1.0. Moreover, the maximum “noise” between pulse beats between individuals varied from a low of −0.119 to a high of −0.340. Thus, there is a large safety margin between the “noise” and the real pulse beats, which is determined at Step 5B. FIG. 17 also allows calculation of the RR values for each two minute measurement cycle. This calculation should include the average RR between pulse peaks, or its standard deviation.

During any extended measurement period—e.g., two minutes—if the time between two pulse beats is twice the value of the average RR, it should be assumed that either the heart has skipped a beat (as occurs in approximately ten percent of healthy population) or that a motion artifact interfered with the pulse beat measurement. If this occurs, at Steps 5C and 5D an artificial pulse beat is inserted half way between the adjacent pulse beats.

In addition, using a Fast Fourier Transform (FFT), the low frequency LF value is determined at Step 6. As previously described, this heart rate variability information can be used as a second regression term or used as a signal to indicate when measurements should be stopped and then resumed during transitions between different sleep states. After the above verification process is completed, at Step 7 the blood glucose value is determined using either linear regression (i.e., Equation 1) or Multiple Linear Regression as previously described.

The invention having been thus described, it will be apparent to those skilled in the art that the same may be varied in many ways without departing from the spirit and scope of the invention. All such variations as would be apparent to those skilled in the art are intended to be covered by the following claims.

1. A method for detecting a potentially undesirable low level of glucose in the blood, comprising the steps of:
taking an initial accurate measurement of blood glucose level;
taking an initial measurement of heart rate within a predetermined amount of time from the taking of said accurate measurement;
periodically monitoring heart rate over a predetermined extended period of time; and
estimating blood glucose level as a function of the periodically monitored heart rate, initial measurement of heart rate, and initial accurate measurement of blood glucose level.

2. The method of claim 1, wherein the step of taking an accurate measurement comprises an invasive drawing of blood.

3. The method of claim 1, wherein the step of estimating comprises using a simple linear regression equation.

4. The method of claim 1, wherein the step of estimating comprises using a multiple linear regression equation.

5. The method of claim 4, wherein said multiple linear regression equation contains a heart rate change variable and a heart rate slow wave activity variable.

6. The method of claim 1, wherein said predetermined amount of time from the taking of said accurate measurement includes a period of time prior to the taking of said accurate measurement.

7. The method of claim 1, wherein the step of periodically monitoring heart rate comprises the step of using an optical measurement instrument to monitor pulse rate.

8. The method of claim 1, wherein the step of estimating blood glucose level comprises the steps of detecting a transition between sleep states and suspending estimation of blood glucose level during such transition.

9. The method of claim 8, wherein the step of detecting a transition comprises measuring heart rate variability.

10. The method of claim 1, further comprising the step of outputting an alarm in the event that blood glucose level estimation results in a blood glucose level value that is undesirably low.

11. A system for detecting a potentially undesirable low level of glucose in the blood, comprising:
an instrument for taking an accurate measurement of blood glucose level;
an instrument for taking an initial measurement of heart rate within a predetermined amount of time from the taking of said accurate measurement and periodically monitoring heart rate over a predetermined extended period of time; and
a device for estimating blood glucose level as a function of the periodically monitored heart rate, initial measurement of heart rate, and accurate measurement of blood glucose level.

12. The system of claim 11, wherein the instrument for taking an accurate measurement comprises an instrument that obtains a sample of blood.

13. The system of claim 11, wherein the device for estimating comprises a data processor.

14. The system of claim 13, wherein the data processor comprises a remote computer.

15. The system of claim 13, wherein the data processor stores a simple linear regression equation for use in blood glucose level estimation.

16. The system of claim 13, wherein the data processor stores a multiple linear regression equation for use in blood glucose level estimation.

17. The system of claim 16, wherein said multiple linear regression equation contains a heart rate change variable and a heart rate slow wave activity variable.

18. The system of claim 11, wherein said predetermined amount of time from the taking of said accurate measurement includes a period of time prior to the taking of said accurate measurement.

19. The system of claim 11, wherein the instrument for periodically monitoring heart rate comprises an optical measurement instrument to monitor pulse rate.

20. The system of claim 11, further comprising an alarm for alerting a monitoring individual in the event that blood glucose level estimation results in a blood glucose level value that is undesirably low.
